(19) United States
(12) Patent Application Publication
(10) Pub. No.: US 2008/0287537 A1
(43) Pub. Date: Nov. 20, 2008

(54) Composition, Particularly a Cosmetic Composition, Comprising ((Dialkylamino)Alkoxy) Ethanol Ester

(75) Inventor: Maria Dalko, Gif S/Yvette (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/579,593

(22) PCT Filed: Apr. 18, 2005
(86) PCT No.: PCT/FR05/00927

§ 371 Date: Aug. 12, 2008

Related U.S. Application Data
(60) Provisional application No. 60/574,594, filed on May 27, 2004.

(30) Foreign Application Priority Data
May 6, 2004 (FR) 0404914

(51) Int. Cl. A61K 31/221; A61K 31/23; C07C 229/22; A61Q 19/08

(52) U.S. Cl. 514/552; 514/546; 560/252

(57) ABSTRACT

The present invention relates to a process for the cosmetic treatment of wrinkled skin, comprising the topical application to the said skin of a composition comprising, in a physiologically acceptable medium, at least one ester of ((dialkylamino)alkoxy)ethanol of formula (I):

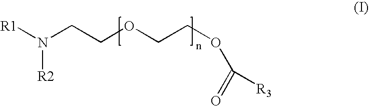

It also relates to novel compounds belonging to the formula (I) and corresponding to the formulae (II) and (III):

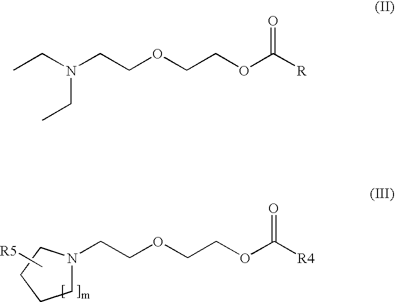

and to the cosmetic compositions comprising them.

The present invention relates to a process for the cosmetic treatment of wrinkled skin comprising the topical application to the said skin of a composition comprising, in a physiologically acceptable medium, at least one ((dialkylamino)alkoxy)ethanol ester of specific formula. It also relates to a novel family of such compounds and to the cosmetic compositions comprising them.

Women, and even men, currently have a tendency to wish to appear young for as long as possible and consequently wish to soften signs of ageing of the skin, which are reflected in particular by wrinkles and fine lines. On that subject, advertising and fashion give instances of products intended to retain a radiant and wrinkle-free skin for as long as possible, these being signs of a young skin, all the more so as the physical appearance affects the mind and/or the morale.

To date, wrinkles and fine lines have been treated using cosmetic products comprising active principles which act on the skin, for example by moisturizing it or by improving its cell replacement or also by promoting the synthesis, or by preventing the decomposition, of the elastic fibres of which skin tissue is composed.

Although these treatments make it possible to act on wrinkles and fine lines due to chronological or intrinsic ageing and on those due to photoageing, they do not have an effect on expression wrinkles, which require operating on the muscular contractile component of the wrinkles present in the skin.

To date, the only means commonly used for acting on the expression wrinkles is botulinum toxin, which is injected in particular into the wrinkles of the glabella, which are wrinkles between the eyebrows (see J. D. Carruters et al., J. Dermatol. Surg. Oncol., 1992, 18, pp. 17-21).

In addition, the Applicant Company has provided various compounds capable of offering a muscle-relaxing effect when they are applied topically to the skin, thus making it possible to act via another route on expression wrinkles. Mention may in particular be made, among these compounds, of calcium channel-associated receptor antagonists (FR-2 793 681), and in particular manganese and its salts (FR-2 809 005) and alverine (FR-2 798 590); and chloride channel-associated receptor agonists, including glycine (EP-0 704 210) and certain Iris pallida extracts (FR-2 746 641).

However, the need remains to have available compounds effective in smoothing out or softening expression wrinkles.

In point of fact, the Applicant Company has discovered, with astonishment, that certain ((dialkylamino)alkoxy)ethanol esters make it possible to satisfy this need.

Certain compounds used according to the invention, namely the ester of coconut fatty acids and of (diethylamino)ethanol ethoxylates, have already been employed in hair hygiene products. However, to the knowledge of the Applicant Company, the suggestion has never been made to use it on the skin of the face, in particular for the purpose of reducing expression wrinkles.

A subject-matter of the present invention is thus a process for the cosmetic treatment of wrinkled skin, in particular the skin of the face and/or forehead, comprising the topical application to the said skin of a composition comprising, in a physiologically acceptable medium, at least one compound chosen from esters of ((dialkylamino)alkoxy)ethanol of formula (I):

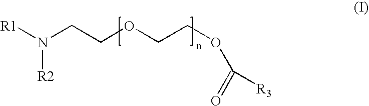

in which:
R₁ and R₂ independently denote: a linear or branched C₁-C₁₀ alkyl or alkenyl group which is optionally substituted by a saturated or unsaturated carbocycle comprising from 5 to 7 carbon atoms; or a saturated or unsaturated carbocycle comprising from 5 to 7 carbon atoms; or R₁ and R₂ form, with the nitrogen atom to which they are connected, a saturated or unsaturated heterocycle comprising from 5 to 6 atoms which is optionally substituted by an aryl group or by a C₁-C₁₈ alkyl group which is optionally substituted by an aryl group;
R₃ denotes an aryl group or a linear, branched or cyclic C₁-C₂₅ alkyl or alkenyl group which are optionally substituted by at least one radical chosen from: a cycloalkyl, phenyl, —OR′, —COOR′ and —NR′R″ radical, where R′ and R″ independently denote a hydrogen atom or a linear or branched C₁-C₆ alkyl or alkenyl group;
n ranges from 1 to 10,
and their addition salts with an acid or a base.

In the formula (I), the alkyl groups can in particular be chosen, as the case may be, from the methyl, ethyl, n-propyl, isopropyl, n-butyl, isobutyl, tert-butyl, pentyl, hexyl, heptyl, octyl, nonyl, decyl, undecyl, dodecyl, myristyl, palmityl and stearyl groups.

In addition, in the context of this patent application, the term “alkenyl” is understood to mean radicals which can comprise one or more conjugated or nonconjugated double bonds. They can in particular be chosen, as the case may be, from the vinyl, allyl, butenyl or pentenyl groups.

The carbocycles can in particular be chosen from the cyclopentyl, cyclohexyl and cycloheptyl radicals, the cyclopentyl and cyclohexyl radicals being preferred.

The nitrogenous heterocycles can be chosen in particular from piperidine, pyrrolidine, piperazine, pyrimidine and morpholine. They can thus comprise, in addition to the nitrogen atom, another nitrogen atom and/or an oxygen atom.

Preference is given, as aryl group, to the use of the phenyl radical.

Mention may be made, as salts of the ester of formula (I), of the salts obtained by addition of the ester of formula (I) with an inorganic acid, chosen in particular from hydrochloric acid, sulphuric acid and phosphoric acid, or with an organic acid, chosen in particular from succinic acid, fumaric acid, lactic acid, glycolic acid, citric acid, tartaric acid, acetic acid and propionic acid. Mention may also be made of the salts obtained by addition of the compound of formula (I) with an inorganic base, such as sodium hydroxide, potassium hydroxide, calcium hydroxide, sodium carbonate, sodium hydrogencarbonate, potassium carbonate, potassium hydrogencarbonate, calcium carbonate or calcium hydrogencarbonate, or with an organic base, such as triethylamine or triethanolamine.

It is clearly understood that the composition used according to the invention can comprise several compounds as defined above, in particular a random mixture of several compounds having different values of n and/or variable alkyl chain lengths for R. This is in particular the case when the ester of formula (I) is synthesized from starting materials of vegetable origin.

The esters of formula (I) can be prepared in particular according to one of the conventional methods well known to a person skilled in the art from the corresponding amino(poly)ethoxyethanol and the corresponding acid or acid chloride.

It is thus possible to react the amino(poly)ethoxyethanol with the acid chloride (route A), with the acid activated by reagents, such as dicyclohexylcarbodiimide (DCC) or carbonyldiimidazole (CDI), for example, (route B) or directly with the carboxylic acid (route C).

To prepare the ester according to route A, it is possible, for example, to dissolve the amino(poly)ethoxyethanol in a nonprotic organic solvent, such as dichloromethane, DMF, dioxane or THF, for example, to add 1 to 10 equivalents (preferably 2 eq.) of organic base, such as pyridine, triethylamine or ethyldiisopropylamine, or of inorganic base, such as sodium bicarbonate, to this solution and then to slowly add 1 to 10 equivalents of acid chloride (preferably 1.2 eq.). The medium is kept stirred for 1 h to 48 h at a temperature of 10° C. to 70° C., the reaction time and the temperature depending on the medium and on the reactivity of the acid chloride used. At the end of the reaction, the product obtained can be purified by silica gel column chromatography, precipitation or recrystallization.

To prepare the ester according to route B, it is possible, for example, to activate the acid functional group in situ with reagents widely known to a person skilled in the art, such as described, for example, on pp. 393-396 in “Advanced Organic Chemistry, Reactions, Mechanisms and Structure”, 4th Edition, by Jerry March, published by Wiley-Interscience, 1992.

To prepare the ester according to route C, it is possible to directly heat the amino(poly)ethoxyethanol in the presence of the carboxylic acid, with or without solvent. The water formed is distilled off directly, in the case of reaction without solvent, or distilled off in the form of an azeotropic mixture with the solvent, such as toluene, or indeed even trapped by a drying agent introduced into the reaction medium, such as a molecular sieve or any other dehydrating agent.

The starting amino(poly)ethoxyethanol is available commercially for R₁═R₂=ethyl or methyl and n equal to 1. When R₁ and R₂ are other than methyl or ethyl groups, the amino(poly)ethoxyethanol can be prepared by reaction of the corresponding secondary amine with commercial 2-(2-chloroethyl)ethanol in methanol or acetonitrile at reflux overnight. The product thus obtained can subsequently be treated and purified on a silica column. For the other values of n, the amino(poly)ethoxyethanol can be synthesized in particular from the corresponding polyol ether and the corresponding dialkylamine according to the protocol described in Application EP-0 300 323.

Processes well known to a person skilled in the art are involved.

According to a first preferred embodiment, the compound according to the invention is such that at least one of the following conditions, and preferably all of these conditions, are satisfied:
R₁ and R₂ are chosen independently from a methyl or ethyl radical;
R₃ is an unsubstituted linear C₂-C₁₉ alkyl or alkenyl radical;
n ranges from 1 to 9; preferably, n is equal to 1, 2, 5 or 6.

According to a first alternative form of this embodiment, the compound according to this embodiment is such that:
R₁ and R₂ are each an ethyl radical;
R₃ is an unsubstituted linear C₅-C₁₇ alkyl or alkenyl radical; and
n is equal to 1.

According to a second alternative form, the compound used according to the invention is such that:
R₁ and R₂ are each an ethyl radical;
R₃ is an unsubstituted linear C₆-C₂₀ alkyl or alkenyl radical; and
n is statistically between 4 and 5.

According to a second embodiment of the invention, the compound of formula (I) is such that:
R₁ and R₂ form, with the nitrogen atom to which they are connected, a piperidine or pyrrolidine ring substituted by a phenyl, benzyl, 2-phenylethyl or 3-phenylpropyl group;
R₃ is an unsubstituted linear C₅-C₁₇ alkyl radical, preferably an undecyl radical; and
n is equal to 1.

The compounds according to this embodiment can be prepared according to a two-stage process comprising:
(a) the reaction of piperidine or pyrrolidine (R₁R₂NH) with 2-(2-chloroethyl)ethanol in methanol or acetonitrile at reflux, for example overnight, and
(b) the reaction of the product obtained in stage (a) with the acyl chloride R₃—CO—Cl in the presence of triethylamine in dichloromethane at ambient temperature, as indicated above (route A).

As will be demonstrated in the examples below, the Applicant Company has demonstrated a dermo-decontracting and muscle-relaxing effect of the compounds according to the invention which makes it possible to envisage their use, more particularly in the smoothing out of expression wrinkles.

Consequently, a subject-matter of the invention is thus the cosmetic use of at least one compound as defined above, in a composition suitable for topical application to the skin, as agent intended to smooth out wrinkles, in particular expression wrinkles.

Furthermore, the Applicant Company has demonstrated that some of the compounds used according to the invention are novel and exhibit an advantageous dermo-decontracting or muscle-relaxing activity.

Another subject-matter of the invention is thus a subfamily of compounds derived from ((dialkylamino)alkoxy)ethanol which are chosen from the esters which correspond to the formula (II) below:

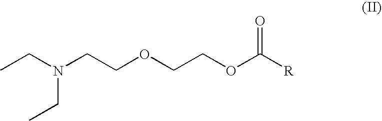

in which:
R denotes an unsubstituted linear C₅-C₁₇ alkyl or alkenyl radical,
and their addition salts with an acid or a base.

These compounds can be prepared as described above.

Examples of esters of formula (II) are such that R is chosen from the n-pentyl, n-heptyl, n-nonyl, n-undecyl, n-tridecyl, n-pentadecyl or n-heptadecyl radicals and the 8,11-heptadecadienyl radical (corresponding to the linoleate). A preferred compound is such that R denotes an n-nonyl radical.

Another subject-matter of the invention is another subfamily of compounds derived from ((dialkylamino)alkoxy)ethanol which are chosen from the esters which correspond to the formula (III) below:

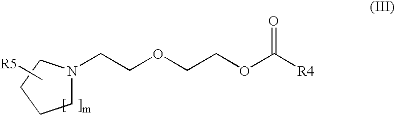

in which:
R₄ denotes a linear, branched or cyclic C₂-C₂₁ alkyl or alkenyl group or an aryl group, which groups can be substituted by at least one radical chosen from: a cycloalkyl radical; a phenyl radical optionally substituted by one or more radicals chosen from OR′, COOR′, linear or branched C₁-C₆ alkyl and CF₃ radicals; an —OR′ radical; a —COOR′ radical; and an —NR′R″ radical; where R′ and R″ independently denote a hydrogen atom or a linear or branched C₁-C₆ alkyl or alkenyl group;
R₅ denotes a phenyl, benzyl, 2-phenylethyl or 3-phenylpropyl group;
m is equal to 1 or 2;
and their addition salts with an acid or a base.

The acid or the base used to salify the esters of formulae (II) and (III) can be any physiologically acceptable acid or base as defined above.

A subject-matter of the present invention is consequently a composition, in particular suitable for topical application to the skin, comprising, in a physiologically acceptable medium, at least one compound chosen from the esters corresponding to the formula (II) or (III) and their addition salts with an acid or a base.

The amount of esters of formula (I), and thus of formula (II) or (III), which can be used according to the invention and/or of their salts depends, of course, on the effect desired and can thus vary within wide limits.

To give an order of magnitude, these compounds can be used in an amount representing from 0.01% to 10% of the total weight of the composition, preferably in an amount representing from 0.05% to 5% of the total weight of the composition, more preferably in an amount representing from 0.1% to 2% of the total weight of the composition.

The composition according to the invention is suitable for topical application to the skin and it thus comprises a physiologically acceptable medium, that is to say a medium compatible with the skin and optionally with its superficial body growths (eyelashes, nails, hair) and/or the mucous membranes. This medium is advantageously cosmetically acceptable, that is to say that it does not result in itching, smarting or redness liable to dissuade the user of the composition and that it exhibits a pleasant appearance, a pleasant smell and a pleasant feel.

This composition can be provided in all the pharmaceutical forms normally used in the cosmetics field and it can in particular be in the form of an optionally gelled solution, of an optionally two-phase dispersion of the lotion type, of an emulsion obtained by dispersion of a fatty phase in an aqueous phase (O/W) or vice versa (W/O), or of a triple (W/O/W or O/W/o) emulsion or of a vesicular dispersion of ionic and/or nonionic type. These compositions are prepared according to the usual methods. It is preferable to use, according to this invention, a composition in the form of an oil-in-water emulsion.

This composition can be more or less fluid and have the appearance of a white or coloured cream, of an ointment, of a milk, of a lotion, of a serum, of a paste or of a foam. It can optionally be applied in the aerosol form. It can also be provided in the solid form, in particular in the stick form. It can be used as care product and/or as makeup product for the skin.

In a known way, the composition used according to the invention can also comprise the adjuvants usual in the cosmetics field, such as hydrophilic or lipophilic gelling agents, hydrophilic or lipophilic active principles, preservatives, antioxidants, solvents, fragrances, fillers, screening agents, pigments, odour absorbers and colouring materials. The amounts of these various adjuvants are those conventionally used in the field under consideration, for example from 0.01 to 20% of the total weight of the composition. These adjuvants, depending on their nature, can be introduced into the fatty phase, into the aqueous phase or into the lipid vesicles. In any case, these adjuvants and their proportions will be chosen so as not to harm the properties desired for the compounds according to the invention.

When the composition used according to the invention is an emulsion, the proportion of the fatty phase can range from 5 to 80% by weight and preferably from 5 to 50% by weight, with respect to the total weight of the composition. The oils, the emulsifiers and the coemulsifiers used in the composition in the emulsion form are chosen from those conventionally used in the field under consideration. The emulsifier and the coemulsifier are present in the composition in a proportion ranging from 0.3 to 30% by weight and preferably from 0.5 to 20% by weight, with respect to the total weight of the composition.

Mention may be made, as oils which can be used in the invention, of mineral oils (liquid petrolatum), oils of vegetable origin (avocado oil, soybean oil), oils of animal origin (lanolin), synthetic oils (perhydro-squalene), silicone oils (cyclomethicone) and fluorinated oils (perfluoropolyethers). Use may also be made, as fatty substances, of fatty alcohols (cetyl alcohol), fatty acids or waxes (carnauba wax, ozokerite).

Mention may be made, as emulsifiers and coemulsifiers which can be used in the invention, for example, of esters of fatty acid and of polyethylene glycol, such as PEG-100 stearate, and esters of fatty acid and of glycerol, such as glyceryl stearate.

Mention may in particular be made, as hydrophilic gelling agents/thickening agents, of carboxyvinyl polymers (carbomer), acrylic copolymers, such as acrylate/alkylacrylate copolymers, polyacrylamides, polysaccharides, natural gums and clays and mention may be made, as lipophilic gelling agents/thickening agents, of modified clays, such as bentones, metal salts of fatty acids and hydrophobic silica.

As active principles, it will be advantageous to introduce, into the composition used according to the invention, at least one compound chosen from: desquamating agents; moisturizing agents; depigmenting or propigmenting agents; antiglycation agents; NO-synthase inhibitors; agents which stimulate the synthesis of dermal or epidermal macromolecules and/or which prevent their decomposition; agents which stimulate the proliferation of fibroblasts and/or of keratinocytes or which stimulate the differentiation of keratinocytes; other muscle-relaxing and/or dermo-decontracting agents; tightening agents; agents for combating pollution or free radicals; agents which act on the microcirculation; agents which act on the energy metabolism of the cells; and their mixtures.

Examples of such additional compounds are: retinol and its derivatives, such as retinyl palmitate; ascorbic acid and its derivatives, such as magnesium ascorbyl phosphate and ascorbyl glucoside; tocopherol and its derivatives, such as tocopheryl acetate; nicotinic acid and its precursors, such as nicotinamide; ubiquinone; glutathione and its precursors, such as L-2-oxothiazolidine-4-carboxylic acid; plant extracts and in particular plant proteins and their hydrolysates, and also phytohormones; marine extracts, such as algal extracts; bacterial extracts; sapogenins, such as diosgenin, and the wild yam extracts comprising the latter; ceramides; hydroxy acids, such as salicylic acid and 5-(n-octanoyl)salicylic acid; resveratrol; oligopeptides and pseudopeptides and their acylated derivatives; manganese and magnesium salts, in particular gluconates; and their mixtures.

As indicated above, the composition according to the invention can also include photoprotective agents active in the UVA and/or UVB regions, in the form of organic or inorganic compounds, the latter optionally being coated in order to render them hydrophobic.

The organic photoprotective agents can be chosen in particular from: anthranilates, in particular menthyl anthranilate; benzophenones, in particular benzophenone-1, benzophenone-3, benzophenone-5, benzophenone-6, benzophenone-8, benzophenone-9, benzophenone-12 and preferably benzophenone-3 (oxybenzone) or benzophenone-4 (Uvinul MS40, available from BASF); benzylidenecamphors, in particular 3-benzylidene camphor, benzylidene camphor sulphonic acid, camphor benzalkonium methosulphate, polyacrylamidomethyl benzylidene camphor, terephthalylidene dicamphor sulphonic acid and, preferably, 4-methylbenzylidene camphor (Eusolex 6300, available from Merck); benzimidazoles, in particular benzimidazilate (Neo Heliopan AP, available from Haarmann and Reimer) or phenylbenzimidazole sulphonic acid (Eusolex 232, available from Merck); benzotriazoles, in particular drometrizole trisiloxane or methylene bis benzotriazolyl tetramethylbutylphenol (Tinosorb M, available from Ciba); cinnamates, in particular cinoxate, DEA methoxycinnamate, diisopropyl methyl cinnamate, glyceryl ethylhexanoate dimethoxycinnamate, isopropyl methoxycinnamate, isoamyl cinnamate and preferably etocrylene (Uvinul N35, available from BASF), octyl methoxycinnamate (Parsol MCX, available from Hoffmann-LaRoche) or octocrylene (Uvinul 539, available from BASF); dibenzoylmethanes, in particular butyl methoxydibenzoylmethane (Parsol 1789); imidazolines, in particular ethylhexyl dimethoxybenzylidene dioxoimidazoline; PABAs, in particular ethyl dihydroxypropyl PABA, ethylhexyl dimethyl PABA, glyceryl PABA, PABA, PEG-25 PABA and preferably diethylhexyl butamido triazone (Uvasorb HEB, available from 3V Sigma), ethylhexyl triazone (Uvinul T150, available from BASF) or ethyl PABA (benzocaine); salicylates, in particular dipropylene glycol salicylate, ethylhexyl salicylate, homosalate or TEA salicylate; triazines, in particular anisotriazine (Tinosorb S, available from Ciba); or drometrizole trisiloxane.

The inorganic photoprotective agents are preferably composed of zinc oxide and/or of titanium dioxide, preferably of nanometric size, optionally coated with alumina and/or with stearic acid.

The composition according to the invention is advantageously intended to be applied to the areas of the face and/or of the forehead marked by expression wrinkles and/or to the people exhibiting expression wrinkles.

The wrinkles concerned are preferably those positioned radially around the mouth and/or eyes, in particular crows feet, and/or situated on the forehead, in particular the glabellar lines situated in the glabella in the space between the eyebrows and/or positioned horizontally on the forehead.

The invention will now be illustrated by the following nonlimiting examples. In these examples, the amounts are shown as percentage by weight.

### EXAMPLES

### Example 1

### Preparation of the Alkyl Esters of ((diethylamino)ethoxy)ethanol

Various compounds according to the invention were prepared according to the synthetic scheme below:

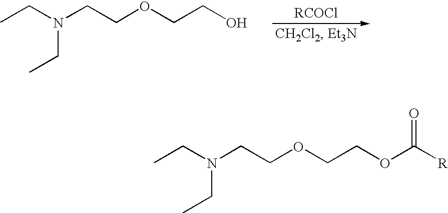

This process comprises the following stages. Commercial ((diethylamino)ethoxy)ethanol is dissolved in dichloromethane. Subsequently, 2.1 equivalents of triethylamine and then, slowly, 1.05 equivalents of acid chloride RCOCl are added and the reaction mixture is left to react at ambient temperature for 20 h. The medium is diluted with dichloromethane and then two aqueous washing operations are carried out. The organic phase is dried over sodium sulphate, filtered and then concentrated to dryness. The residue obtained is purified by a silica column or by precipitation.

The reactant (acyl chloride) used and the results obtained are collated in the table below.

| | | | ¹H NMR |
|---|---|---|---|
| R | Yield | Appearance | 500 MHz |
| C₂H₅ | 56% | Slightly brown oil | Conforms |
| C₇H₁₅ | 60% | Slightly brown oil | Conforms |
| C₉H₁₉ | 72% | Slightly brown oil | Conforms |
| C₁₁H₂₃ | 82% | Slightly brown oil | Conforms |
| C₁₃H₂₇ | 80% | Slightly brown oil | Conforms |
| C₁₅H₃₁ | 80% | Slightly brown oil | Conforms |
| C₁₇H₃₅ | 44% | Slightly brown oil | Conforms |
| C₂₁H₄₃ | 44% | Beige waxy solid | Conforms |

### Example 2

### Preparation of the Ester of Coconut Fatty Acids and of ethoxylated (diethylamino)ethanol (n=1-9)

This mixture of compounds can be prepared by direct heating of a mixture of aminoethanol ethoxylates in the presence of coconut fatty acids, with or without solvent. The water formed is distilled off directly, in the case of reaction without solvent, or distilled off in the form of an azeotropic mixture with the solvent, such as toluene, indeed even trapped by a drying agent introduced into the reaction medium, such as a molecular sieve or any other dehydrating agent. The mixture of compounds obtained has an amine number of 1.89 (meq/g) and the following structure:

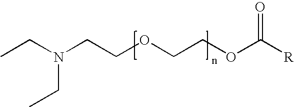

where R corresponds to a coconut fatty acids residue and n is statistically between 4 and 5.

### Example 3

### Demonstration of the Calcium-Inhibiting Effect of the Compounds According to the Invention

In order for a substance to be recognized as a calcium-channel inhibitor, it must be able to reduce the intracellular calcium concentration or reduce the binding of calcium to intracellular proteins, such as, for example, calmodulin, such as is described in particular by Galizzi, J. P. et al., J. Biol. Chem., 1987, 262 p. 6947; by Y. Okamiya et al., Eur. J. Pharmacol., 1991, 205, p. 49; by J. A. Wagner et al., J. Neurosci., 1988, 8, p. 3354; by H. R. Lee et al., Life Sci., 1984, 35, p. 721; by Schoemaker H. and Lauger S., Eur. J. Pharmacol., 1985, 111, p. 273 or by I. J. Reynolds et al., J. Pharmacol. Exp. Ther., 1986, 237, p. 731.

The Applicant determined the IC_{50Ca2+} for inhibition of calcium flux of three compounds according to the invention using the protocol shown in these documents. The results are given in Table 1 below. IC_{50Ca2+} represents the concentration which inhibits release of Ca²⁺ by 50%.

| | IC_{50Ca2+} in μM | |
|---|---|---|
| | on cells resulting | |
| | from HFDa skin | on SKNSH nerve |
| Compounds tested | fibroblasts | cells |
| Compound of Example 2 | 36 | — |
| ((Diethylamino)ethoxy)ethyl | — | 26 |
| decanoate | | |
| ((Diethylamino)ethoxy)ethyl | 13 | 11 |
| laurate | | |
| ((Diethylamino)ethoxy)ethyl | 14 | 24 |
| palmitate | | |
| ((Diethylamino)ethoxy)ethyl | — | 37 |
| stearate | | |

It emerges, from this table, that the compounds according to the invention are indeed calcium-channel inhibitors.

It is deduced, from these tests and from the teaching of Application EP-1 053 745, that they exhibit a high probability of having a beneficial effect on wrinkles and in particular expression wrinkles.

### Example 4

### Demonstration of an Inhibiting Effect on L-Type Calcium Channels

### a) Protocol

The ability of the ((diethylamino)ethoxy)ethyl laurate of Example 1 (at 1 μM in DMSO) and of the mixture of compounds of Example 2 (at 10 μM in DMSO) to competitively inhibit the fixing of L-type calcium-channel agonists was evaluated.

The studies are carried out starting from rat cerebral cortex (isolated membranes exhibiting L-type calcium channels at their surface) homogenates according to the method described by Reynolds I. J. et al., 1986, J. Pharmacol. Exp. Ther., 237, p. 731.

The experimental conditions are as follows:

| | | | Non- | Incu- | |
|---|---|---|---|---|---|
| Test | Ligand | Conc. | specific | bation | Detection |
| Ca²⁺channel | [³H]-(−)- | 0.5 nM | D600 | 60 min/ | Scintil- |
| (L, verapamil | D888 | | (10 μM) | 22° C. | lation |
| site) | | | | | counting |

D888, which is [³H]-(−)-desmethoxyverapamil, acts as radiolabelled specific ligand and
D600, which is (±)-methoxyverapamil hydrochloride, acts as reference molecule.

The specific binding of a ligand (labelled D888) to the receptors (L-type calcium channels, verapamil site) is defined as the difference between the total binding and the non-specific binding determined in the presence of an excess of cold (nonradioactive) ligand. The results are expressed as percentage of inhibition of the specific binding of the control in the presence of the compound tested.

### b) Results

The compound of Example 1 inhibits the specific binding of the control to the calcium channels by 31%. The mixture of compounds of Example 2 inhibits the specific binding of the control to the calcium channels by 95%. It is deduced, from this test and from the teaching of Application EP-1 053 745, that these compounds exhibit a high probability of having a beneficial effect on wrinkles and in particular expression wrinkles.

### Example 5

### Demonstration of the Muscle-Relaxing Effect of the Compounds According to the Invention

The mixture of compounds of Example 2 was tested on a model of nerve/muscle coculture which makes it possible to recreate a motor arc by innervating human striated muscle cells with explants of rat embryo spinal ganglia and spinal cord.

This test is predictive of an antiwrinkle effect, as has been demonstrated by the Applicant Company in the case of diazepam, which inhibited the contractions of the muscle fibres in this model and the antiwrinkle activity of which has been demonstrated in vivo.

### a) Protocol

Human muscle cells, resulting from samples of striated muscles from a healthy donor, are inoculated in wells with a cross section of 1.8 cm² (24-well culture dishes). After culturing for 10 days, these cells form a monolayer and fuse. At this stage, spinal cord explants from rat embryos aged 13 days comprising the spinal ganglia are deposited on the culture.

The growth of the neurites is visible outside the spinal cord explant after culturing for one day. The first contractions of the muscle fibres are observed after coculturing for 5 to 6 days and, after 3 weeks, all the muscle fibres in the vicinity of the explants are contracting.

The cocultures are used after 21 days, when the muscle fibres are striated and have mature differentiated neuromuscular junctions.

A muscle fibre having regular contractions (at least 60 contractions per minute) is then selected from three different culture wells and the number of contractions is counted over 30 seconds. The compound tested, diluted in DMSO, is subsequently incubated in these wells for 60 seconds at a concentration of 10, 50 and 100 μM. At the end of incubation, the number of contractions is again counted over 30 seconds. The test is carried out in triplicate.

### b) Results

The mixture of compounds of Example 2 blocks the contractions of the three muscle fibres referred to, at the concentrations of 10, 50 and 100 μM.

These compounds can thus be used to relax the lines of the face and to smooth out expression wrinkles.

### Example 6

### Cosmetic Composition

This composition is prepared in the way conventional to a person skilled in the art. The amounts shown are as percentages by weight.

| | |
|---|---|
| ((Diethylamino)ethoxy)ethyl decanoate | 1% |
| Propylene glycol isostearate | 13% |
| Polyethylene glycol (8 EO) | 5% |
| Propylene glycol | 3% |
| Pentylene glycol | 3% |
| Glyceryl stearate and polyethylene glycol | 5% |
| (100 EO) stearate | |
| Oxyethylenated (20 EO) sorbitan monostearate | 0.5% |
| Oxyethylenated (20 EO)/oxypropylenated | 1% |
| (5 PO) cetyl alcohol | |
| Gelling agent | 0.5% |
| C₁₂₋₁₅ alkyl benzoates | 4% |
| Ethanol | 3% |
| Sodium hydroxide | 0.12% |
| Preservatives | 0.7% |
| Water | q.s. for 100% |

This fluid is intended to be applied once or twice daily to the face and the forehead in order to tone down expression wrinkles.

1. Process for the cosmetic treatment of wrinkled skin, comprising the topical application to said skin of a composition comprising, in a physiologically acceptable medium, at least one compound chosen from esters of ((dialkylamino)alkoxy)ethanol of formula (I): 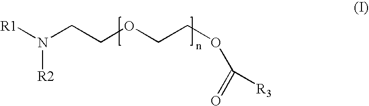
in which:
R₁ and R₂ independently denote: a linear or branched C₁-C₁₀ alkyl or alkenyl group which is optionally substituted by a carbocycle comprising from 5 to 7 carbon atoms; a saturated or unsaturated carbocycle comprising from 5 to 7 carbon atoms; or R₁ and R₂ form, with the nitrogen atom to which they are connected, a saturated or unsaturated heterocycle comprising from 5 to 6 atoms which is optionally substituted by an aryl group or by a C₁-C₁₈ alkyl group which is optionally substituted by an aryl group;
R₃ denotes an aryl group or a linear, branched or cyclic C₁-C₂₅ alkyl or alkenyl group which is optionally substituted by at least one radical chosen from: a cycloalkyl, phenyl, —OR′, —COOR′ and —NR′R″ radical, where R′ and R″ independently denote a hydrogen atom or a linear or branched C₁-C₆ alkyl or alkenyl group;
n ranges from 1 to 10;
and their addition salts with an acid or a base.

2. Process according to claim 1, wherein the salt of the ester of formula (I) is obtained by addition with an inorganic acid selected from the group consisting of hydrochloric acid, sulphuric acid and phosphoric acid.

3. Process according to claim 1, wherein the salt of the ester of formula (I) is obtained by addition with an organic acid selected from the group consisting of succinic acid, fumaric acid, lactic acid, glycolic acid, citric acid, tartaric acid, acetic acid and propionic acid.

4. Process according to claim 1, wherein the salt of the ester of formula (I) is obtained by addition with an inorganic base selected from the group consisting of sodium hydroxide, potassium hydroxide, calcium hydroxide, sodium carbonate, sodium hydrogencarbonate, potassium carbonate, potassium hydrogencarbonate, calcium carbonate and calcium hydrogencarbonate.

5. Process according to claim 1, wherein the salt of the ester of formula (I) is obtained by addition with an organic base selected from the group consisting of triethylamine and triethanolamine.

6. Process according to claim 1, wherein said compound is such that at least one of the following conditions is satisfied:
R₁ and R₂ are chosen independently from a methyl or ethyl radical;
R₃ is an unsubstituted linear C₂-C₁₉ alkyl or alkenyl radical;
n ranges from 1 to 9.

7. Process according to claim 6, wherein said compound is such that:
R₁ and R₂ are each an ethyl radical;
R₃ is an unsubstituted linear C₅-C₁₇ alkyl or alkenyl radical; and
n is equal to 1.

8. Process according to claim 6, wherein said compound is such that:
R₁ and R₂ are each an ethyl radical;
R₃ is an unsubstituted linear C₆-C₂₀ alkyl or alkenyl radical; and
n is statistically between 4 and 5.

9. Process according to claim 1, wherein the said compound is such that:
R₁ and R₂ form, with the nitrogen atom to which they are connected, a piperidine or pyrrolidine ring substituted by a phenyl, benzyl, 2-phenylethyl or 3-phenylpropyl group;
R₃ is an unsubstituted linear C₅-C₁₇ alkyl radical, preferably a dodecyl radical; and
n is equal to 1.

10. Process according to claim 1, wherein said compound represents from 0.1 to 2% of the total weight of the composition.

11. Process according to claim 1, wherein said composition additionally includes at least one compound selected from the group consisting of desquamating agents; moisturizing agents; depigmenting or propigmenting agents; antiglycation agents; NO-synthase inhibitors; agents which stimulate the synthesis of dermal or epidermal macromolecules and/or which prevent their decomposition; agents which stimulate the proliferation of fibroblasts and/or of keratinocytes or which stimulate the differentiation of keratinocytes; other muscle-relaxing and/or dermo-decontracting agents; tightening agents; agents for combating pollution or free radicals; agents which act on the microcirculation; agents which act on the energy metabolism of the cells; and their mixtures.

12. Process according to claim 1, wherein said composition is applied to the skin of the face and/or of the forehead.

13. Process according to claim 12, wherein said composition is applied to the areas of the face and/or of the forehead marked by expression wrinkles and/or to the people exhibiting expression wrinkles.

14. Process according to claim 12, wherein said composition is applied to the wrinkles positioned radially around the mouth and/or eyes and/or horizontally on the forehead and/or situated in the space between the eyebrows.

15. A method of smoothing out wrinkles comprising applying the composition according to claim 1 to skin in need thereof.

16. The method according to claim 15, wherein said wrinkles are expression wrinkles.

17. Compounds derived from ((dialkylamino)alkoxy)ethanol which are chosen from the esters which correspond to the formula (II) below: 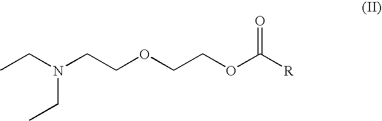
in which:
R denotes an unsubstituted linear C₅-C₁₇ alkyl or alkenyl radical,
and their addition salts with an acid or a base.

18. Compounds according to claim 17, wherein R denotes an n-nonyl radical.

19. Compounds derived from ((dialkylamino)alkoxy)ethanol which are chosen from the esters which correspond to the formula (III) below: 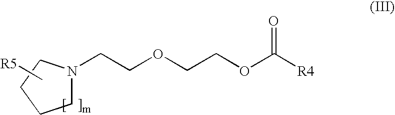
in which:
R₄ denotes a linear, branched or cyclic C₂-C₂₁ alkyl or alkenyl group or an aryl group, which groups can be substituted by at least one radical chosen from: a cycloalkyl radical; a phenyl radical optionally substituted by one or more radicals chosen from OR′, COOR′, linear or branched C₁-C₆ alkyl and CF₃ radicals; an —OR′ radical; a —COOR′ radical; and an —NR′R″ radical; where R′ and R″ independently denote a hydrogen atom or a linear or branched C₁-C₆ alkyl or alkenyl group;
R₅ denotes a phenyl, benzyl, 2-phenylethyl or 3-phenylpropyl group;
m is equal to 1 or 2;
and their addition salts with an acid or a base.

20. Composition comprising at least one of the compounds according to claim 17 in a physiologically acceptable medium.

21. Composition according to claim 20, wherein it is suitable for topical application to the skin.

22. The process according to claim 9, wherein R₃ is an undecyl radical.

23. Composition comprising at least one of the compounds according to claim 19 in a physiologically acceptable medium.
